(19) United States
(12) Patent Application Publication
(10) Pub. No.: US 2015/0335714 A1
(43) Pub. Date: Nov. 26, 2015

(54) USE OF HUMAN CHORIONIC GONADOTROPIN TO INHIBIT DEVELOPMENT OR MINIMIZE SEVERITY OF CEREBRAL PALSY AND/OR ITS CO-MORBIDITIES

(71) Applicants: Tammy Z. MOVSAS, (US); Rafael GALINDO, (US); ZIETCHICK RESEARCH INSTITUTE, LLC., Southfield, MI (US); WASHINGTON UNIVERSITY IN ST. LOUIS, Louis, MO (US)

(72) Inventors: Tammy Z. Movsas, Southfield, MI (US); Rafael Galindo, Kirkwood, MO (US)

(21) Appl. No.: 14/762,983

(22) PCT Filed: Jan. 23, 2014
(86) PCT No.: PCT/US14/12675

§ 371 Date: Jul. 23, 2015

Related U.S. Application Data
(60) Provisional application No. 61/756,534, filed on Jan. 25, 2013.

(51) Int. Cl. A61K 38/24

(52) U.S. Cl. CPC A61K 38/24

(57) ABSTRACT

The invention described herein relates generally to the use of human chorionic gonadotropin (hCG) or a subunit or variant thereof, in neurodevelopmental disorders. In particular, the present invention relates to the use of hCG and related compounds as described herein for treating or reducing the likelihood of cerebral palsy and its co-morbidities in a patient or subject including a neonate, an infant or a child.

### RELATED APPLICATIONS

This application claims the benefit of priority of U.S. provisional application Ser. No. U.S. 61/756,534, filed Jan. 25, 2013, entitled “USE OF HUMAN CHORIONIC GONADOTROPIN TO INHIBIT DEVELOPMENT OR MINIMIZE SEVERITY OF CEREBRAL PALSY”, the entire contents of which application is incorporated by reference herein.

### FIELD OF THE INVENTION

The invention described herein relates generally to the use of human chorionic gonadotropin (hCG) or a subunit or variant thereof, in neurodevelopmental disorders. In particular, the present invention relates to the use of hCG as described herein for treating or reducing the likelihood of cerebral palsy (CP) and/or its co-morbidities in a neonate, infant or child.

### BACKGROUND OF THE INVENTION

In recent years, hCG has been found to have many important, diverse functions during pregnancy. To name a few, hCG promotes blood vessel formation in uterine vasculature, blocks maternal immune or macrophage action against foreign invading placental cells and ensures that uterine growth parallels fetal growth.¹⁻³ Back in early 1980's, hCG receptors were identified in multiple fetal organs including brain, kidney, lung and colon⁴⁻⁵ thus suggesting a diverse role for hCG in fetal growth and differentiation.⁶⁻⁹ However, little progress has been made over the years in defining the neurodevelopmental role of this hormone.⁶⁻⁹ Studies have shown that hCG can cross the blood-brain barrier, and that hCG receptors exist in both fetal and infant brains.⁶⁻⁹ Furthermore, studies in vitro have demonstrated that hCG can decrease neuronal cell death and promote the outgrowth of fetal rat brain neuronal cell processes.⁷ In addition, adult injury models suggest that hCG may have neuroprotective properties in vivo. For example, hCG has been shown to promote the proliferation of neuronal stem cells and has been recently proposed as a potential therapeutic strategy for the treatment of adult stroke.¹⁰⁻¹¹ Despite the above evidence, prior to the filing of this patent application, no one has previously considered the novel use of hCG as a preventative agent for CP and its co-morbidities.

CP is a collective name given to a range of conditions resulting in physical disability caused by brain injury which occurs either perinatally (before, at or around the time of birth), or during the first few years of an infant's life while the baby's brain is still developing. Term and premature infants are at a significant risk for CP due to the vast array of risk factors that may negatively affect neuronal health including neonatal cerebral hypoxia-ischemia, stroke, intraparenchymal or intraventricular hemorrhage and neonatal sepsis to name a few. Symptoms of CP can be very different between people with this group of disorders.

### Symptoms may:

Be very mild or very severe
Only involve one side of the body or both sides
Be more pronounced in either the arms or legs, or involve both the arms and legs

Symptoms are usually seen before a child is 3 years old, and sometimes they may be present or identified at birth. Parents may notice that their child is delayed in reaching for objects, and in developmental stages such as sitting, rolling, crawling, or walking.

There are several different types of CP, including spastic, dystonic, dyskinetic, ataxic, hypotonic, and mixed. Symptoms of spastic CP, the most common type, include:
Muscles that are very tight and do not stretch. They may tighten up even more over time.
Abnormal gait and postural movements: arms tucked in toward the sides, knees crossed or touching, legs make “scissors” movements, walk on the toes
Joints are tight and do not open up all the way (called joint contracture)
Muscle weakness or loss of movement in a group of muscles (paralysis)
The symptoms may affect one arm or leg, one side of the body, both legs, or both arms and legs
   At the present time, therapeutic hypothermia (head or body cooling) is the only effective intervention for neonatal brain hypoxia-ischemia, which is a major risk factor for CP. With no current cure, physicians mainly attempt to prevent CP by improving obstetric care and minimizing the key CP pre- and perinatal risk factors such as prematurity, low birth weight and perinatal infection. Magnesium sulfate has recently been shown to decrease the risk of CP in very preterm infants, although it has not yet been implemented into clinical practice. Therefore, magnesium sulfate's real life effectiveness is not yet known.

### PRIOR ART

European patent application EP 0605501, entitled “Use of presynaptic neurotoxin for the treatment of cerebral palsy” provides for the use of a presynaptic neurotoxin (for example a bacterial neurotoxin such as botulinum toxin A) for the treatment of CP in juvenile patients up to 6 years in age. This type medicament helps the promotion of normal muscle growth in a juvenile patient before the patient has completed its growing period and fixed contracture has occurred suffering from dynamic contractures due to CP.

### OBJECTS OF THE INVENTION

It is an object of the invention to provide methods of treating CP in a neonate, infant and/or child in need by administering effective amounts of hCG or a subunit or variant thereof to the neonate, infant and/or child, or alternatively, to the mother pregnant with a fetus likely to be diagnosed with CP, or alternatively, to the mother pregnant with a fetus likely to be diagnosed with CP as well as to the neonate and/or infant after birth.

It is an additional object of the invention to provide methods for inhibiting the severity or reducing the likelihood of CP in a neonate and/or infant by administering to the neonate and/or infant at risk for CP or alternatively, to the mother pregnant with a fetus at risk for CP, an effective amount of hCG or a subunit or variant thereof or alternatively, to the mother pregnant with a fetus at risk for CP, an effective amount of hCG or a subunit or variant thereof as well as to the neonate and/or infant after birth

It is an additional object of the invention to provide methods of treating (by eliminating and/or reducing the severity of) one or more co-morbidities (i.e., secondary effects) that can accompany CP in a neonate, infant and/or child by administering effective amounts of hCG or a subunit or variant thereof to the neonate, infant and/or child in need, or alternatively, to a mother pregnant with a fetus likely to be diagnosed with CP or further alternatively, to the mother pregnant with a fetus likely to be diagnosed with CP as well as to the neonate and/or infant after birth.

It is an additional object of the invention to provide methods for inhibiting or reducing the likelihood of the co-morbidities that accompany CP in a neonate, infant or child by administering to the neonate, infant and/or child at risk for CP or alternatively, to the mother pregnant with a fetus at risk for CP, an effective amount of hCG or a subunit or variant thereof or alternatively, to the mother pregnant with a fetus at risk for CP, an effective amount of hCG or a subunit or variant thereof as well as to the neonate and/or infant after birth

It is an object of the invention to provide methods of treating cerebral white matter injury, (the most common substrate of CP) in a neonate, infant and/or child by administering effective amounts of hCG or a subunit or variant thereof to the neonate, infant and/or child or alternatively, to the mother pregnant with a fetus likely to be diagnosed with white matter injury or alternatively, to the mother pregnant with a fetus likely to be diagnosed with CP as well as to the neonate and/or infant after birth.

It is an additional object of the invention to provide methods for inhibiting the severity or reducing the likelihood of white matter injury (which is the most common substrate or precursor of CP in preterm neonates and/or infants/children up to about 4-6 years of age and includes leukomalacia as well as other forms of white matter disease) in a neonate and/or infant/child up to about 4-6 years of age by administering to the neonate and/or infant/child at risk for CP or alternatively, to the mother pregnant with a fetus at risk for injury to the cerebral white matter, an effective amount of hCG or a subunit or variant thereof or alternatively, to the mother pregnant with a fetus at risk for white matter injury, including leukomalacia or other forms of white matter disease in the neonate, an effective amount of hCG or a subunit or variant thereof as well as to the infant after birth.

Any one or more of these and/or other objects of the invention may be readily gleaned from a description of the invention which follows.

### BRIEF DESCRIPTION OF THE FIGURES

FIG. 1 shows the amino acid sequence of hCG alpha (92 amino acids) and B-subunits (145 amino acids). Digits indicate amino acid residue positions and N and O indicate the positions of N- and O-linked oligosaccharides¹

FIG. 2 shows the structures of O-linked hexa- and tris-saccharide and N-linked Bi (biantennary) and Tri (triantennary) oligosaccharides attached to regular (non-hyperglycosylated) hCG, hyperglycosylated hCG and hyperglycosylated hCG free Beta. NeuAc is N-acetylneuraminic acid or sialic acid, GalNAc in n-acetylgalactosamine, Gal is galactose, GlcNAc is N-acetylglucosamine, Man is mannose and Fuc is fucose).

FIG. 3 shows the outline of the structures of the 15 common hCG variants present in serum and urine samples in either pregnancy, gestational trophoblastic disease or other malignancy. Numbers refer to subunit poplypeptide amino acid numbers (as in 1 and 145 in the 145 amino acide long beta-subunit). 0 refers to the O-linked and N to the N-linked oligosaccharides. 00 and NN refer to large or hyperglycosylated oligosaccharides. The Greek letters for alpha and beta represent the alpha and beta subunit respectively. Beta-CTP is the C-terminal segment (resideues 93-145) on the regular of hyperglycosylated hCG Beta-subunit.¹

FIG. 4, Table 1 shows the relationship between maternal hCG levels and CP. The consistent inverse relationship between maternal hCG levels and CP Risk (i.e. higher hCG levels is associated with lower CP risk) provides further evidence of hCG's ability to inhibit the development of CP pursuant to the present invention. The references for Table 1 appear after references for the text of the application in the present specification.

FIG. 5 shows the effect of a single intraperitoneal hCG (1500 IU/kg) injection on striatal and hippocampal injury from term-equivalent neonatal hypoxia-ischemia. A. is a histogram showing the percent volume loss in PBS-vs hCG-treated term equivalent mice newborns in the hippocampus (Mean±SEM; 39±2.7% vs. 26±6.5%) and striatum (Mean±SEM; 23±4.4% vs. 13±2.9%). B. shows representative coronal sections of the typical type of injury observed (L; left, R; right) in the striatum (Str) and hippocampus (Hipp) from each treatment group.

FIG. 6 shows that a single (1500 IU/kg) intraperitoneal administration of hCG at postnatal day 6 results in a 1.5 fold increase in VEGF mRNA levels in the neonatal murine cerebral cortex 24 hrs after hormone injection (***p<0.001 by unpaired t-test; N=6).

### SUMMARY OF THE INVENTION

The use of hCG (including but not limited to the 15 variants of hCG and its subunits described herein) pertains to developing a hCG-containing composition or medicament to be administered in therapeutic doses to a mother before or during birth and/or to a neonate and/or infant and/or child after birth to prevent incidence or limit severity of CP or its co-morbidities (such as epilepsy, behavioral, intellectual and language abnormalities) or to reduce the likelihood or limit the severity of cerebral white matter injury (which is the most common substrate or precursor of CP in preterm infants). While not being limited by way of theory, compositions according to the present invention may function to prevent brain injury and/or aid in recovery from brain injury. In additional embodiments, the present composition would be administered to pregnant mothers whose offspring would be at increased risk for CP and/or to the neonates and/or infants who are at increased risk for CP. Routes of administration may include but are not limited to: intrathecal, intravenous, parenteral, intraperitoneal, oral, topical.

### DETAILED DESCRIPTION OF THE INVENTION

The following terms shall be used to describe the present invention. In the absence of a specific definition set forth herein, the terms used to describe the present invention shall be given their common meaning as understood by those of ordinary skill in the art.

The term “patient” or “subject” is used throughout the specification to describe an animal, generally a human, but in certain instances, a domesticated animal such as cattle, pigs, horses, sheep, cats and dogs to whom hCG or a subunit and/or variant thereof is administered to effect an intended result in that patient or subject. For purposes of certain embodiments according to the present invention where administration of hCG or a subunit and/or a variant thereof is administered to a mother carrying a fetus at risk for CP, the patient or subject is considered the mother and fetus together. In most instances, the term patient or subject refers to a human patient or subject, including a pregnant mother (and the fetus carried by the pregnant mother) a neonate (i.e., a newborn from just after birth to about 1 month old, including a preterm neonate), an infant (from about 1 month old to up to about 2 years old) and/or a child (up to about 10 years of age, but often up to about 4-6 years of age).

The term “effective amount” is used throughout the specification to describe an amount of the present composition (hCG and/or a subunit or variant thereof, including hyperglycosylated and nicked variants of hCG as otherwise described herein, which terms include pharmaceutically acceptable salts of these compounds/compositions) which are used to effect an intended result when used in the method of the present invention. In numerous aspects of the present invention the term effective amount is used in conjunction with the treatment of a patient, generally a neonate and/or an infant or child suffering from or at risk of CP to treat, to inhibit the severity of CP, ameliorate the symptomatology, including co-morbidities and/or secondary effects of CP or reduce the likelihood of CP in said patient. In other embodiments, the term effective is used in conjunction with the administration of hCG or a subunit or variant thereof to a mother pregnant with a fetus at risk for CP. In other aspects, the term effective amount simply refers to an amount of an agent which produces a result which is seen as being beneficial or useful in methods according to the present invention.

The term effective amount with respect to the presently described compounds and compositions is used throughout the specification to describe that amount of the compound or composition according to the present invention which is administered to a mammalian patient or subject, especially including a human patient or subject, who is afflicted with signs and or symptoms of CP or who is at risk for CP, to inhibit the severity of CP, including symptomatology and co-morbidities and/or secondary effects of CP, as well as reducing the likelihood that a patient or subject at risk for CP will become afflicted with CP.

The term “human chorionic gonadotropin hormone” or “hCG” refers to a heterogeneous molecule which is a glycoprotein composed of 2 dissimilar subunits, α- and β-subunits, coded by separate genes on separate chromosomes, held together by covalent bonding and charge interactions. The hCG α-subunit is composed of 92 amino acids and contains 2 N-linked oligosaccharides. In contrast, the hCG β-subunit is composed of 145 amino acids and contains 2 N-linked and 4 O-linked oligosaccharides. The 8 oligosaccharide side chains comprise >30% of the molecular weight of hCG, making it an exceptionally highly glycosylated glycoprotein.

hCG while similar in structure to luteinizing hormone (LH), naturally exists in multiple hormonal and non-endocrine agents, rather than as a single molecule like LH and the other glycoprotein hormones. These include regular hCG, hyperglycosylated hCG and the free beta-subunit of hCG, as well as the hyperglycosylated free beta-subunit of hCG. Regular hCG predominates in all normal and abnormal pregnancies and it is the principal hCG variant produced through the bulk period of pregnancy. Regular hCG is also normally produced by the pituitary gland at the time of LH peaks and in menopausal women. Of the 15 hCG variants, 5 are natural products made by the human body and the other 10 variants are degradation products from macrophage cleavage and cleavage by proteases in the circulation and the kidney. In the present invention, all hCG variants, including hCG, hyperglycosylated hCG, free hCG β-subunit, hyperglycoslated hCG free β-subunit, which are preferred, may be used in the present invention. In particularly preferred embodiments, hyperglycosylated hCG and regular hCG may be preferably administered, with regular hCG being preferred.

Peptide variants, cleaved or nicked forms of hCG, free subunits of hCG, and fragments of hCG are all viewed as variants pursuant to the present invention and in most instances detectable in serum and urine samples during pregnancy. Oligosaccharide variants reflect availability of sugar substrates, and general cellular metabolism, expression of different glycosyltransferases by cells. It has long been recognized that the hCG molecule, particularly the β-subunit of hCG, produced in choriocarcinoma (trophoblastic cancer) and testicular germ cell cancer, migrates slower than hCG β-subunit standards on electrophoresis gels and elutes earlier than hCG β-subunit standards from gel filtration columns because of the increased glycosylation reflective of hyperglycosylation. This is believed to be due to the presence of large oligosaccharides on the hCG β-subunit. Further studies with lectins and structural studies have indicated the presence of larger or more complex oligosaccharides on choriocarcinoma hCG. There has been demonstrated a major difference between the 4 O-linked oligosaccharides on hCG in choriocarcinoma and normal pregnancy hCG. The hCG from normal pregnancies primarily contains a mixture of tri- and tetrasaccharides, with approximately 0-14% hexasaccharide. In contrast, hyperglycosylated hCG (choriocarcinoma hCG) contains over 50% of the hexasaccharide structure.

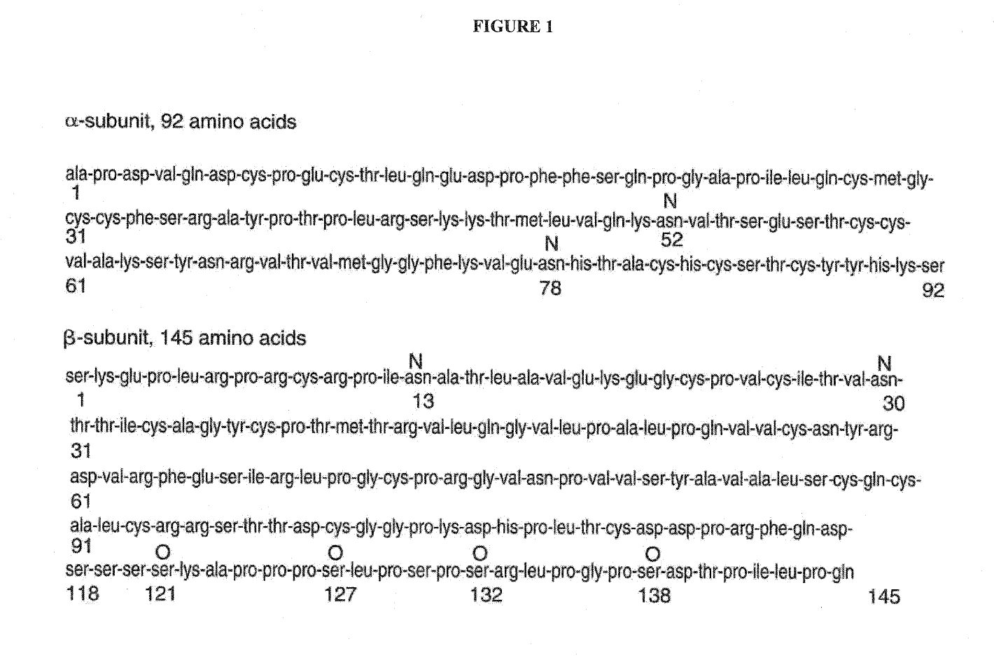

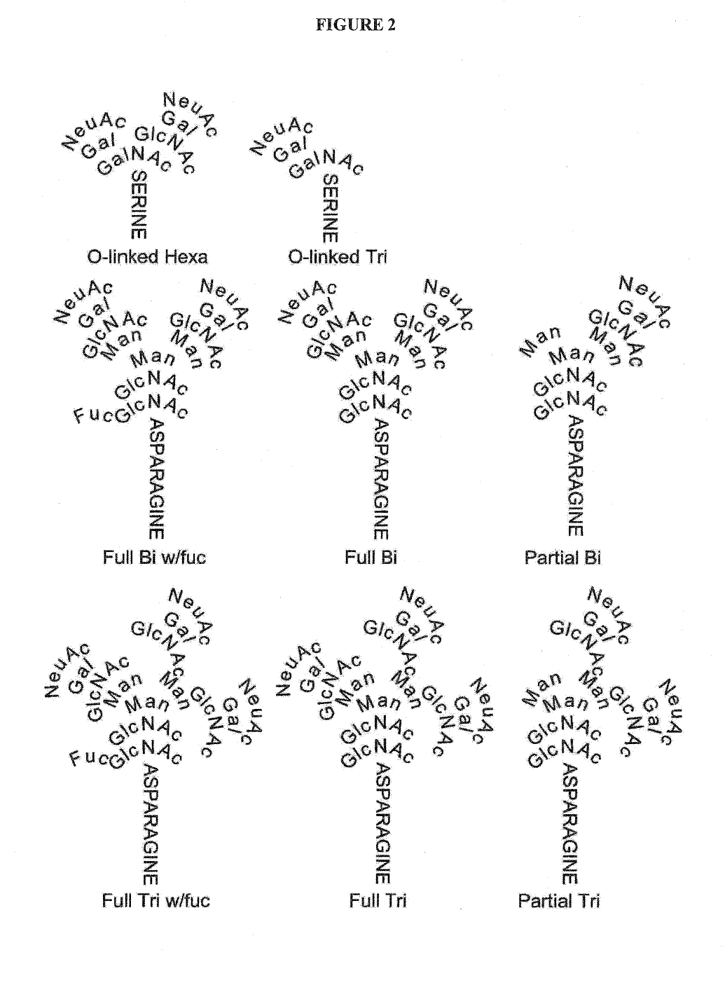

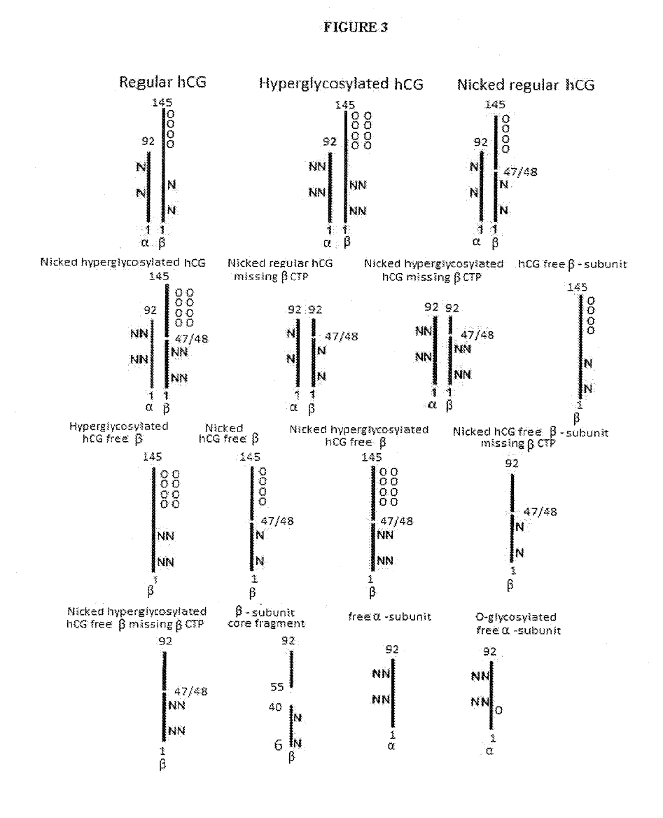

It has been shown that the difference in the 4 O-linked oligosaccharides is the principal variation between hyperglycosylated hCG (choriocarcinoma hCG) and regular hCG (pregnancy hCG). While first trimester normal pregnancy urine hCG contains approximately 12.3 to 19% (mean=15.6%) hexasaccharide structures, choriocarcinoma urine hCG (hyperglycosylated hCG) contained 48 to 100% hexasaccharide structures. A smaller change is observed in α-subunit and β-subunit N-linked oligosaccharides (from an average of 6.8% and 14% triantennary structures in first trimester pregnancy to 9.8% and 51% triantennary structures in choriocarcinoma, on α- and β-subunit respectively. We call the hCG produced in choriocarcinoma hyperglycosylated hCG or H-hCG because of the large size due to the overly large sugar units. Assays and monoclonal antibodies are available which detect each of these hCG compounds. Structures of a number of hCG variants which find use in the present invention are depicted in attached FIGS. 1-3. While hCG, its subunits and variants thereof, including pharmaceutically acceptable salts, may be used in the present invention, preferably regular hCG, hyperglycosylated hCG, free β-subunit hCG and hyperglycosylated free β-subunit hCG are used in the present invention, with regular hCG (which term includes any appropriate salt form which can be placed in solution and administered to a neonate, an infant/child less than 4 years of age and preferably less than 2 years of age, or a pregnant mother carrying a fetus).

The term “cerebral palsy” (CP) is used to describe a group of non-progressive disorders of movement and posture caused by abnormal development of, or damage to, motor control centers of the brain. CP is caused by events before, during, or after birth. The abnormalities of muscle control that define CP are often accompanied by other neurological and physical abnormalities such as, but not limited to, cognitive impairment, seizures and difficulties with vision, speech, hearing and swallowing. CP does not include conditions due to inheritably progressive disease or degeneration of the brain. For this reason, CP is also referred to as static (non-progressive) encephalopathy (disease of the brain). Also excluded from CP are any disorders of muscle control that arise in the muscles themselves and/or in the peripheral nervous system (synapses and nerves outside the brain and spinal cord).

“Co-morbidities” (secondary effects) that may accompany CP include but are not limited to the following:

### Brain and Nervous System Co-Morbidities

Chronic cognitive impairment
Expressive and/or receptive language impairment
Partial or complete hearing or vision loss
Seizures (Epilepsy)
Chronic pain
Behavior difficulties
Neurobehavioral disorders such as attention deficits and hyperactivity.
Autism Spectrum Disorder (Double the number preterm children with autism have cerebral palsy compared to those without autism)¹²

### Eating and Digestive Co-Morbidities

Difficulty sucking or feeding in infants, or chewing and swallowing in older children and adults
Vomiting or constipation

### Other Co-Morbidities: Increased Drooling, Slow Growth, Irregular Breathing, Urinary Incontinence

Neonatal stroke and perinatal brain hypoxia-ischemia are examples of two major risk factors for CP development; Brain injury may be caused, for example, by a hypoxic-ischemic event during pregnancy or after birth or by trauma during delivery. Sometimes, clinically relevant injuries to the developing brain are the result of a combination of least two insults. For example, a predisposing factors (pre-conditioning cytotoxic factors, such as chronic ethanol consumption), may increase the susceptibility of the developing brain to a second, injury-producing (precipitating), event (such as hypoxia-ischemia), even when each of these insults may not have been of great enough magnitude to have caused injury in and of themselves.¹³ At other times, the cause of the brain injury that led to CP is unknown.

The most common underlying substrate (precursory or feature) of CP in premature infants is injury and loss to the cerebral white matter (leukomalacia), and in particular arising from a condition termed periventricular leukomalacia (PVL) which is defined by two components: (1) a “focal” necrotic component in the periventricular region of the brain and (2) a “diffuse” component characterized by reactive gliosis in the surrounding white matter.¹⁴ Preterm infants are at a particular risk for white matter injury due to the relatively high susceptibility of axonal supporting and myelin forming cells (i.e oligodendrocytes) to hypoxia and/or ischemia. Though white matter injury commonly occurs in very preterm infants, it can also occur in late preterm and term infants.

Some of the major causes of white matter injury in the neonate are:
(1) Cerebral ischemia in the premature infant with cerebral vascular immaturity coupled with the propensity for impaired vascular autoregulation.
(2) Infection in the mother (and fetus) during pregnancy and/or in the infant after birth alone or coupled with cerebral ischemia (An ischemic insult which may not have been of sufficient severity to produce brain injury may do so when accompanied or preceded by infection)
(3) Intraventricular and/or cerebral intraparenchymal hemorrhage precipitated with the fragile nature of premature cerebral vessels and immature vascular autoregulation.

Focal necrotic lesions of leukomalacia involving nerve cells or axon pathways of the corticospinal tract correlate well with the later development of cerebral palsy (motor deficits) and the diffuse white matter lesions correlate with the development of cognitive and behavioral abnormalities (these are examples of co-morbidities of CP but can also occur independently of the occurrence of CP).¹⁴

Below are examples of some types of patients who may be at increased risk for CP (and/or its associated co-morbidites, a “patient or subject at risk”) and thus may benefit from treatment according to the present invention. Patients may fall into more than one category from the list below (Please note that patients that may benefit from the present invention for CP prevention are not limited to the list below).
All patients who had been born very preterm (<31 week of gestational age)
Patients who were exposed to infections during pregnancy (i.e. bacterial or viral infections)
Any term or preterm patients with inflammatory/infectious illnesses (such as necrotizing enterocolitis or neonatal sepsis) after birth
Any term patient with inflammatory/infectious illness after birth
Patients at risk for white matter injury Patients with radiographic or ultrasonic evidence of periventricular leukomalacia or other type of white matter abnormality
Patients or pregnant mothers with infections during pregnancy
Patients who experienced hypoxic-ischemic events during pregnancy, at birth or after birth.
Patients with hypoxic ischemic encephalopathy
Patients with intracranial hemorrhage
Patients with brain infections (encephalitis, meningitis, congenital infections)
Patients with head injury or birth trauma
Patients who may have been exposed to a cytotoxic agent (such as chronic alcohol exposure during pregnancy) which may make the patient more vulnerable to brain injury from mild hypoxic insults (which commonly occur during pregnancy but may not be clinically detected)

The terms “treat”, “treating”, and “treatment”, are used synonymously to refer to any action providing a benefit to a subject or patient at risk for or afflicted with a disease, including improvement in the condition through lessening, inhibition, amelioration or suppression of at least one symptom or sign, delay in progression of the disease, prevention (reduction in the likelihood) or delay in the onset of the disease.

Treatment, as used herein, encompasses both prophylactic and therapeutic treatment. Compounds according to the present invention can, for example, be administered prophylactically to a subject/patient in advance of the occurrence of disease to reduce the likelihood of that disease. Prophylactic administration is effective to reduce or decrease the likelihood of the subsequent occurrence of disease or co-morbidities or secondary effects of disease in the mammal, or decrease/inhibit the severity of disease that subsequently occurs. Alternatively, compounds or compositions according to the present invention can, for example, be administered therapeutically to a subject or patient that is already afflicted b_{y} disease. In one embodiment of therapeutic administration, administration of the present compounds is effective to inhibit the severity of the disease state or condition, ameliorate some or all of the symptomology of the disease state or condition or reduce the likelihood that a subject or patient will contract the disease state or condition as otherwise described herein.

The term “pharmaceutically acceptable”, which term includes “pharmaceutically acceptable salts” as used herein means that the compound or composition is suitable for administration to a subject to achieve the treatments described herein, without unduly deleterious side effects in light of the severity of the disease state and/or condition and the necessity of the treatment. The term “inhibit” as used herein refers to the partial or complete elimination of a potential effect.

As used herein, “alleviating a cerebral palsy symptom” means reducing the severity of the symptom and/or co-morbidity/secondary effect. Thus, a disease or disorder is “alleviated” if the severity of a symptom and/or co-morbidity/secondary effect of the disease or disorder, the frequency with which such a symptom and/or co-morbidity/secondary effect is experienced by a patient, or both, are reduced.

As used herein, “treating cerebral palsy” means reducing the severity and/or inhibiting the worsening of CP and/or its co-morbidities/secondary effects and/or symptoms consistent with treatment.

A “prophylactic” treatment is a treatment administered to a subject who does not exhibit signs of a disease or exhibits only early signs of the disease or condition or symptoms of the disease or condition for the purpose of decreasing the risk of (i.e., decreasing the likelihood of) developing CP or a co-morbidity. Typically, the agents of prophylaxis for use in the present invention exhibit a high therapeutic index and, in particular, low toxicity and are generally the same as those which are used in therapy of Cerebral Palsy.

A “therapeutic” treatment is a treatment administered to a subject who exhibits signs of CP for the purpose of diminishing or eliminating those signs or, alternatively, is a treatment administered to a subject in need for amelioration of the severity of the disease state or condition or its symptoms.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “prevention” when used in context shall mean “reducing the likelihood” or in isolated instances, preventing a condition or disease state from occurring as a consequence of administration or concurrent administration of one or more compounds or compositions according to the present invention, alone or in combination with another agent which may be administered to ameliorate or eliminate the disease or condition or one or more symptoms thereof. It is noted that prophylaxis will rarely be 100% effective; consequently the terms prevention and reducing the likelihood are used to denote the fact that within a given population of patients or subjects, administration with compounds according to the present invention will reduce the likelihood or inhibit a particular condition or disease state (in particular, the worsening of a disease state such as deteriorating nervous system function or other accepted indicators of disease progression in the case of inflammatory and neurologic diseases) from occurring.

The present invention relates to the administration of an identified compound(s) in a pharmaceutical composition to practice the methods of the invention, the composition comprising the compound or an appropriate derivative or fragment of the compound and a pharmaceutically acceptable carrier, additive or excipient. As used herein, the term “pharmaceutically acceptable carrier, additive or excipient” means a chemical composition with which an appropriate compound as hCG or a subunit or variant thereof as otherwise described herein either alone or in combination, can be used to administer the appropriate compound(s) to a patient or subject in therapeutic methods according to the present invention.

Pharmaceutical compositions according to the present invention may be administered to deliver an effective dose of between 1 ng/kg/day and 100 mg/kg/day of hCG and/or a subunit or variant thereof to the patient or subject in need of therapy as otherwise described herein. These compositions according to the present invention are generally formulated in the presence of a pharmaceutically acceptable carrier, additive or excipient. Pharmaceutically acceptable carriers, additives and excipients which are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey). The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, intraperitoneal, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Pharmaceutical compositions that are useful in the methods of the invention may be administered systemically in oral solid formulations, ophthalmic, suppository, aerosol, topical or other similar formulations. In addition to the compound such as heparin sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer, for example, peptides, fragments, or derivatives, and/or a nucleic acid encoding the same according to the methods of the invention.

Compounds which are identified using any of the methods described herein may be formulated and administered to a subject/patient, especially a mammal such as a human, for the treatment and/or prevention of CP (or its co-morbidities) as described herein.

The invention encompasses the preparation and use of pharmaceutical compositions comprising a compound useful for the treatment and/or reducing the likelihood of CP (or its co-morbidities/secondary effects) as otherwise described herein. Such a pharmaceutical composition may comprise the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit. Such methods are well known in the art.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Thus, the present invention contemplates a veterinary application. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant domestic mammals such as cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise less than 0.1% to 100% (w/w) active ingredient.

In addition to the active ingredient, a pharmaceutical composition of the invention may further comprise one or more additional pharmaceutically active agents, including other agents which may be used to ameliorate the symptomatology of CP (or its co-morbidities/secondary effects). Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc. Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide for a pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethyl cellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C. and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application into a vein, by application of the composition through a tissue-penetrating non-surgical wound, and the like. In particular, parenteral administration is contemplated to include, but is not limited to, intravenous, intrathecal, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) forin for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% by weight active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity as, for example, an inhaler. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65′F at atmospheric pressure. Generally the propellant may constitute 50 to 99.9% (w/w) of the composition, and the active ingredient may constitute about 0.1 to 20% (w/w) of the composition. The propellant may further comprise additional ingredients such as a liquid non-Ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as in ethylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nose.

Formulations suitable for nasal administration may, for example, comprise from about as little as 0.1% (w/w) and as much as 100% (w/w) of the active ingredient, and they may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (W/W) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “pharmaceutically acceptable additives” include, but are not limited to, one or more of the following: surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additives” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of subject and, e.g. the type of cancer and disease state being treated or the circumstances in which a subject presents for fertility enhancement, the age of the subject, the route of administration and the relative therapeutic index.

The compound can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

In general, water, suitable oil, saline, aqueous dextrose (glucose), and related sugar solutions and glycols such as propylene glycol or polyethylene glycols are suitable carriers for parenteral solutions. Solutions for parenteral administration contain the immunogen optionally along with suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

It will be recognized by one of skill in the art that the various embodiments of the invention as described above relating to specific methods of treating Cerebral Palsy (or its co-morbidities) or ameliorating the disease state and/or condition or its symptoms may relate within context to the treatment or resolution of symptomatology not specifically mentioned herein, or may involve different circumstances of symptomatology. Thus, it should not be construed that embodiments described herein for specific treatments mentioned herein do not apply to other symptoms of CP.

### Kits for Inhibiting Development of Cerebral Palsy

The method of the invention includes a kit comprising a hCG or a subunit or variant thereof and instructional material which describes administering the active agent or a composition comprising the active agent to a cell or a subject/patient, especially a mammal, including a human. This should be construed to include other embodiments of kits that are known to those skilled in the art, such as a kit comprising a (preferably, sterile) solvent suitable for dissolving or suspending the composition of the invention prior to administering the compound to a patient or subject. Preferably the patient or subject is a human, including a neonate, infant or pregnant woman.

As used herein, “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

The following examples are provided to more fully describe the present invention. The examples are not be construed as limiting the present invention in any way or manner.

### Example 1

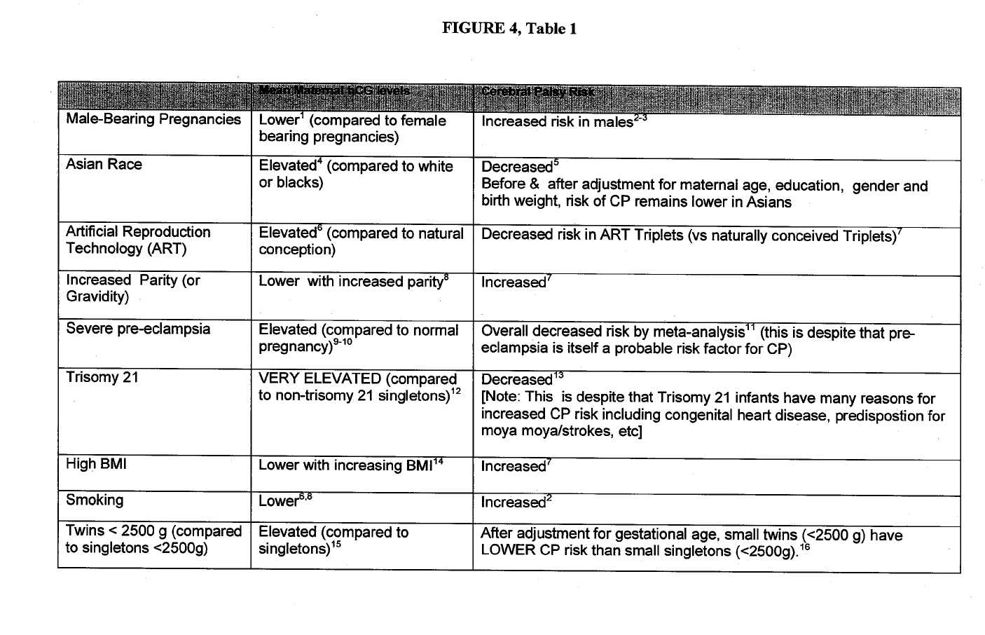

Against the backdrop of the present invention, by integrating information from several different studies in the medical literature, the present inventors were able to indirectly demonstrate and confirm an inverse relationship between maternal hCG levels and CP risk (i.e. higher hCG levels is associated with lower cerebral palsy risk). FIG. 4, table 1, shows the relationship between maternal hCG levels and CP. Thus, against the backdrop of the present invention, the medical literature confirms support of the inventor's theory that hCG, its subunits and/or its variants may be implicated in inhibiting the development CP. However, it is important to note that no prior study either mentions or directly examines this relationship In other words, the present inventors are the first to discover this relationship through extensive reading of the medical literature and integration of scientific laboratory research results which support the thesis and examples of the present invention. In other words, the inventors show in Table 1 that the offspring from several different groups of women who have higher mean hCG levels during pregnancy (such as Asian women or women bearing female fetuses) have lower CP risk.

### Example 2

Perinatal hypoxia-ischemia is one of the greatest risk factors for both CP and for white matter cerebral injury (which is the most common underlying substrate of CP in preterm infants). We demonstrate the neuroprotective role of hCG as CP preventative agent on the injured developing brain utilized the well-studied modified Levine mouse model of neonatal brain hypoxia ischemia (HI).¹⁵ The experiment supporting this invention were designed and carried out by the current inventors in the laboratory of Dr. Holtzman in the Dept of Neurology at Washington University School of Medicine. The injury paradigm that was used consists of ligating the left carotid artery in the term-equivalent mouse (postnatal day 7) and then subjecting the neonatal pup to a hypoxic condition for a predetermined period of time (8% Oxygen for 45 minutes). This model produces consistent and reproducible injury to the ipsilateral ligated side in brain regions that are commonly associated with birth asphyxia in human newborns such as the hippocampus, cerebral cortex and striatum.¹⁶⁻²⁹ Furthermore, this form of murine neonatal injury has demonstrated to result in similar long-term neurocognitive and motor deficits to those seen in children affected with CP.^{30-41,20,24,26-27,42-47} Quantification of the extent of neuroanatomical injury is made by comparing the area of injury in the lesioned ipsilateral-ligated side to the contralateral unlesioned brain in the same animal.

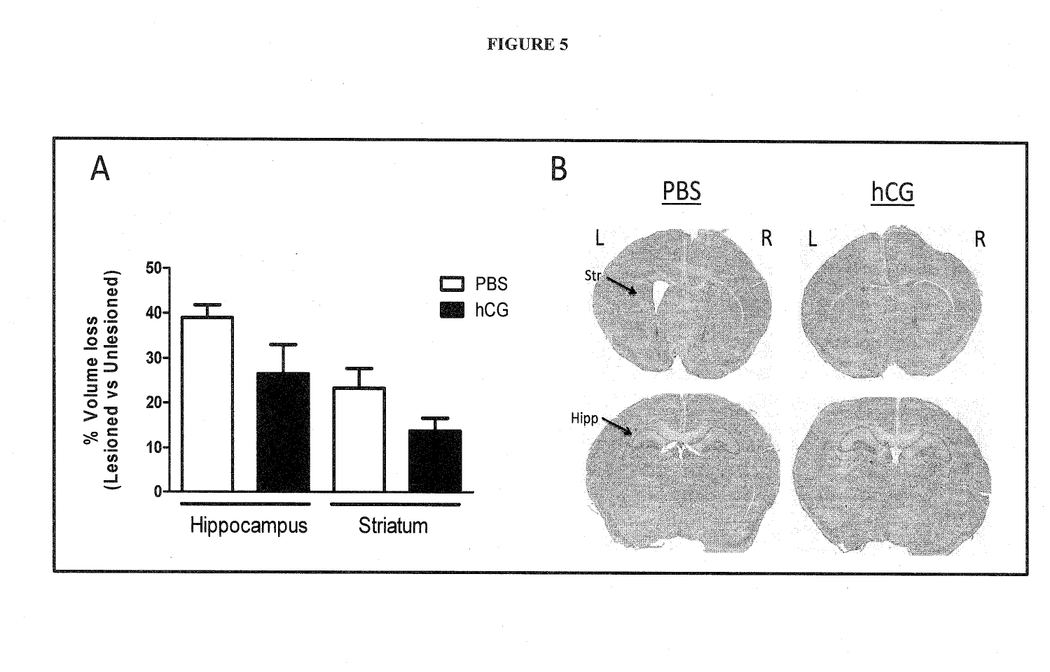

A single 1500 IU/kg intraperitoneal injection of hCG (Sigma-Aldrich, St. Louis Mo.) was administered approximately 15 hours prior to carotid ligation. Phosphate buffered saline (PBS)-injected littermates were used as vehicle controls. PBS (N=6) and hCG-injected (N 6) neonatal pups had the carotid ligation on the same day and they were exposed to the hypoxic chamber at the same time. We chose a single intraperitoneal (IP) dose prior to exposure to the injury paradigm to minimize potential toxic additive effects of hCG taking into account an estimated hCG half-life of 33 hrs. A concentration of 1500 IU/kg of hCG was chosen as a dose that has been previously utilized safely for the study of this hormone's neuroprotective effects in murine models of adult stroke.¹⁰ As demonstrated in FIG. 5, the mean percent volume loss in hippocampal and striatal tissue in hCG-treated neonates was approximately 40% less to those of PBS-treated littermate controls. Unpaired one-tailed t-test showed a p=0.054 in the hippocampus and p=0.051 in the striatal region, bordering on statistical significance set at p<0.05. These results strongly suggest the potential of hCG-dependent neuroprotection from neonatal HI and HI contributes to the development of CP, to white matter injury (a common precursor of CP) and to the co-morbidities/secondary effects of CP.

Proposed Mechanism 1 for the Use of hCG (Including but not Limited to any of its 15 Variants) to Inhibit or Reduce the Likelihood of the Development of CP (and its Co-Morbidities) or Inhibit and/or Ameliorate the Symptomology Associated with Cerebral Palsy:

Without being limited by way of theory, and in light of the evidence accumulated which suggested that hCG is useful in the inhibition and/or treatment of CP, a mechanism for hCG activity is proposed. Notwithstanding the elucidation of this proposed mechanism, without being limited by way of theory, the following proposed mechanism is provided:

### hCG May Protect Against Cerebral Palsy by Upregulating Neural VEGF Expression

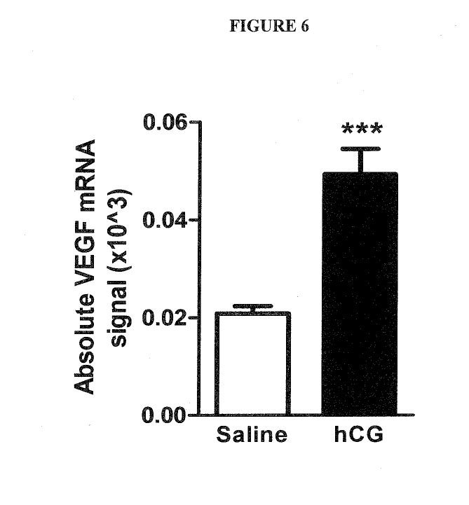

Since hCG is a key regulator of VEGF in the uterine vasculature^{2,48}, and since it has the ability to cross the blood-brain barrier, the present inventors propose that an important mechanism by which hCG may confer neuroprotection is by upregulating neural VEGF levels. With that said, there is ample evidence that VEGF has neuroprotective properties in the developing brain against the effects of hypoxia ischemia.^{44-46,49-58} For example, VEGF has been shown to protect the neonatal rodent cerebral cortex against glutamate-dependent excitotoxicity, a destructive physiological process triggered by the effects of neonatal HI.⁴⁹ Furthermore, VEGF-transfected neural stem cells have been shown to prevent or slow down the brain damage that is associated with CP in newborn rats.⁵⁰ Therefore, we examined whether a single 1500 IU/kg intraperitoneal administration of hCG at postnatal day 6 results in an increase in the expression of VEGF in the neonatal brain 24 hours after hCG administration. As shown in FIG. 6. intraperitoneal administration of hCG increased VEGF mRNA levels by 1.5 fold compared to saline vehicle-treated littermates. This finding supports the neuroprotective role and one of the possible neuroprotective mechanisms of hCG.

### Example 3

hCG Protects Premature Infants from White Matter Injury.

Premature infants are at high risk for CP due to relative vulnerability of the white matter to injury as compared to the term neonate.⁵⁹⁻⁶² Injury to the white matter in the preterm neonate is not only caused by decreases in cerebral oxygenation and/or blood perfusion, but also by other pro-inflammatory processes that result in the activation of astrocytes and microglia⁶². Furthermore, the premature neonate is at particular risk for CP due to the heighten vulnerability to pro-inflammatory conditions such as sepsis and necrotizing enterocolitis among others⁵⁹⁻⁶⁰. Therefore, agents that decrease systemic or central nervous system inflammation and/or ameliorate sepsis are likely good candidates for the prevention of CP in this population. hCG has been to shown to have potent favorable immunosuppressive effects and it has also been shown to ameliorate septic shock in mice and rhesus monkeys⁶³. Consequently, hCG may confer neuroprotection in the ‘at risk’ premature neonate, in part, by decreasing systemic inflammation. In addition, hCG has been shown to activate the secretion of nitric oxide (NO) from activated microglia in vitro⁶⁴. In turn, NO production in the injured brain is known to increase collateral blood flow in affected brain regions resulting in concomitant reductions in amount of neonatal brain injury⁶⁵⁻⁶⁷. Therefore, hCG may not only provide neuroprotection by increasing VEGF expression (as described in Example 2), by also by decreasing neuroinflammation and increasing the production of NO.

### REFERENCES FOR SPECIFICATION TEXT

1. Cole L A. New discoveries on the biology and detection of human chorionic gonadotropin. Reprod Biol Endocrinol. 2009; 7:8.
2. Reisinger K, Baal N, McKinnon T, Munstedt K, Zygmunt M. The gonadotropins: tissue-specific angiogenic factors? Mol Cell Endocrinol. Apr. 15, 2007; 269(1-2):65-80.
3. Sun P D, Davies D R. The cystine-knot growth-factor superfamily. Annu Rev Biophys Biomol Struct. 1995; 24:269-291.
4. Goldsmith P C, McGregor W G, Raymoure W J, Kuhn R W, Jaffe R B. Cellular localization of chorionic gonadotropin in human fetal kidney and liver. J Clin Endocrinol Metab. September 1983; 57(3):654-661.
5. Abdallah M A, Lei Z M, Li X, et al. Human fetal nongonadal tissues contain human chorionic gonadotropin/luteinizing hormone receptors. J Clin Endocrinol Metab. February 2004; 89(2):952-956.
6. Lei Z M, Rao C V, Kornyei J L, Licht P, Hiatt E S. Novel expression of human chorionic gonadotropin/luteinizing hormone receptor gene in brain. Endocrinology. May 1993; 132(5):2262-2270.
7. al-Hader A A, Tao Y X, Lei Z M, Rao C V. Fetal rat brains contain luteinizing hormone/human chorionic gonadotropin receptors. Early Pregnancy. December 1997; 3(4):323-329.
8. A A A L-H, Lei Z M, Rao C V. Neurons from fetal rat brains contain functional luteinizing hormone/chorionic gonadotropin receptors. Biol Reprod. May 1997; 56(5):1071-1076.
9. AA AL-H, Lei Z M, Rao C V. Novel expression of functional luteinizing hormone/chorionic gonadotropin receptors in cultured glial cells from neonatal rat brains. Biol Reprod. February 1997; 56(2):501-507.
10. Belayev L, Khoutorova L, Zhao K L, Davidoff A W, Moore A F, Cramer S C. A novel neurotrophic therapeutic strategy for experimental stroke. Brain Res. Jul. 14, 2009; 1280:117-123.
11. Cramer S C, Fitzpatrick C, Warren M, et al. The beta-hCG+erythropoietin in acute stroke (BETAS) study: a 3-center, single-dose, open-label, noncontrolled, phase Ila safety trial. Stroke. May 2010; 41(5):927-931.
12. Kuzniewicz M W, Wi S, Qian Y, Walsh E M, Armstrong M A, Croen L A. Prevalence and neonatal factors associated with autism spectrum disorders in preterm infants. J Pediatr. January 2014; 164(1):20-25.
13. Genetta T, Lee B H, Sola A. Low doses of ethanol and hypoxia administered together act synergistically to promote the death of cortical neurons. J Neurosci Res. January 2007; 85(1):131-138.
14. Kinney H C. The near-term (late preterm) human brain and risk for periventricular leukomalacia: a review. Semin Perinatol. April 2006; 30(2):81-88.
15. Rice J E, 3rd, Vannucci R C, Brierley J B. The influence of immaturity on hypoxic-ischemic brain damage in the rat. Annals of neurology. February 1981; 9(2):131-141.
16. Carloni S, Balduini W. Experimental models of hypoxic-ischemic encephalopathy: hypoxia-ischemia in the immature rat. Curr Protoc Toxicol. February 2008; Chapter 11:Unit11 15.
17. Northington F J. Brief update on animal models of hypoxic-ischemic encephalopathy and neonatal stroke. Ilar J. 2006; 47(1):32-38.
18. Aden U, Dahlberg V, Fredholm B B, Lai U, Chen Z, Bjelke B. MRI evaluation and functional assessment of brain injury after hypoxic ischemia in neonatal mice. Stroke. May 2002; 33(5):1405-1410.
19. Cengiz P, Kleman N, Uluc K, et al. Inhibition of Na+/H+ exchanger isoform 1 is neuroprotective in neonatal hypoxic ischemic brain injury. Antioxid Redox Signal. May 15, 2011; 14(10):1803-1813.
20. Hutton L C, Ratnayake U, Shields A, Walker D W. Neuropathology and functional deficits in a model of birth asphyxia in the precocial spiny mouse (Acomys cahirinus). Dev Neurosci. 2009; 31(6):523-535.
21. Im S H, Yu J H, Park E S, et al. Induction of striatal neurogenesis enhances functional recovery in an adult animal model of neonatal hypoxic-ischemic brain injury. Neuroscience. Aug. 11, 2010; 169(1):259-268.
22. Liu D, Guo H, Griffin J H, Fernandez J A, Zlokovic B V. Protein S confers neuronal protection during ischemic/hypoxic injury in mice. Circulation. Apr. 8, 2003; 107(13):1791-1796.
23. Loren D J, Seeram N P, Schulman R N, Holtzman D M. Maternal dietary supplementation with pomegranate juice is neuroprotective in an animal model of neonatal hypoxic-ischemic brain injury. Pediatr Res. June 2005; 57(6):858-864.
24. McAuliffe J J, Miles L, Vorhees C V. Adult neurological function following neonatal hypoxia-ischemia in a mouse model of the term neonate: water maze performance is dependent on separable cognitive and motor components. Brain Res. Nov. 6, 2006; 1118(1):208-221.
25. Northington F J, Chavez-Valdez R, Graham E M, Razdan S, Gauda E B, Martin U. Necrostatin decreases oxidative damage, inflammation, and injury after neonatal HI. J Cereb Blood Flow Metab. January 2011; 31(1):178-189.
26. Ten V S, Bradley-Moore M, Gingrich J A, Stark R I, Pinsky D J. Brain injury and neurofunctional deficit in neonatal mice with hypoxic-ischemic encephalopathy. Behav Brain Res. Oct. 17, 2003; 145(1-2):209-219.
27. Ten V S, Wu E X, Tang H, et al. Late measures of brain injury after neonatal hypoxia-ischemia in mice. Stroke. September 2004; 35(9):2183-2188.
28. Verghese P B, Sasaki Y, Yang D, et al. Nicotinamide mononucleotide adenylyl transferase 1 protects against acute neurodegeneration in developing CNS by inhibiting excitotoxic-necrotic cell death. Proc Natl Acad Sci USA. Nov. 22, 2011; 108(47):19054-19059.
29. West T, Atzeva M, Holtzman D M. Pomegranate polyphenols and resveratrol protect the neonatal brain against hypoxic-ischemic injury. Dev Neurosci. 2007; 29(4-5):363-372.
30. Almli C R, Levy T J, Han B H, Shah A R, Gidday J M, Holtzman D M. BDNF protects against spatial memory deficits following neonatal hypoxia-ischemia. Exp Neurol. November 2000; 166(1):99-114.
31. Balduini W, De Angelis V, Mazzoni E, Cimino M. Long-lasting behavioral alterations following a hypoxic/ischemic brain injury in neonatal rats. Brain Res. Mar. 24, 2000; 859(2):318-325.
32. Bona E, Hagberg H, Loberg E M, Bagenholm R, Thoresen M. Protective effects of moderate hypothermia after neonatal hypoxia-ischemia: short- and long-term outcome. Pediatr Res. June 1998; 43(6):738-745.
33. Bona E, Johansson B B, Hagberg H. Sensorimotor function and neuropathology five to six weeks after hypoxia-ischemia in seven-day-old rats. Pediatr Res. November 1997; 42(5):678-683.
34. Chou I C, Trakht T, Signori C, et al. Behavioral/environmental intervention improves learning after cerebral hypoxia-ischemia in rats. Stroke. September 2001; 32(9):2192-2197.
35. Ford L M, Sanberg P R, Norman A B, Fogelson M H. MK-801 prevents hippocampal neurodegeneration in neonatal hypoxic-ischemic rats. Arch Neurol. October 1989; 46(10):1090-1096.
36. Ikeda T, Mishima K, Yoshikawa T, et al. Selective and long-term learning impairment following neonatal hypoxic-ischemic brain insult in rats. Behav Brain Res. Jan. 8, 2001; 118(1):17-25.
37. Jansen E M, Low W C. Long-term effects of neonatal ischemic-hypoxic brain injury on sensorimotor and locomotor tasks in rats. Behav Brain Res. August 1996; 78(2):189-194.
38. Liu X H, Eun B L, Barks J D. Platelet-activating factor antagonist BN 50730 attenuates hypoxic-ischemic brain injury in neonatal rats. Pediatr Res. June 2001; 49(6):804-811.
39. Tomimatsu T, Fukuda H, Endoh M, et al. Effects of neonatal hypoxic-ischemic brain injury on skilled motor tasks and brainstem function in adult rats. Brain Res. Feb. 1, 2002; 926(1-2):108-117.
40. Wagner B P, Nedelcu J, Martin E. Delayed postischemic hypothermia improves long-term behavioral outcome after cerebral hypoxia-ischemia in neonatal rats. Pediatr Res. March 2002; 51(3):354-360.
41. Young R S, Kolonich J, Woods C L, Yagel S K. Behavioral performance of rats following neonatal hypoxia-ischemia. Stroke. November-December 1986; 17(6):1313-1316.
42. Ten V S, Bradley-Moore M, Gingrich J A, Stark R I, Pinsky D J. Brain injury and neurofunctional deficit in neonatal mice with hypoxic-ischemic encephalopathy. Behavioural brain research. Oct. 17, 2003; 145(1-2):209-219.
43. Schlager G W, Griesmaier E, Wegleiter K, et al. Systemic G-CSF treatment does not improve long-term outcomes after neonatal hypoxic-ischaemic brain injury. Exp Neurol. July 2011; 230(1):67-74.
44. Zheng X R, Zhang S S, Yang Y J, et al. Adenoviral vector-mediated transduction of VEGF improves neural functional recovery after hypoxia-ischemic brain damage in neonatal rats. Brain Res Bull. Mar. 16, 2010; 81(4-5):372-377.
45. Feng V, Rhodes P G, Bhatt A J. Hypoxic preconditioning provides neuroprotection and increases vascular endothelial growth factor A, preserves the phosphorylation of Akt-Ser-473 and diminishes the increase in caspase-3 activity in neonatal rat hypoxic-ischemic model. Brain Res. Apr. 14, 2010; 1325:1-9.
46. Fan X, Heijnen C J, van der Kooij M A, Groenendaal F, van Bel F. The role and regulation of hypoxia-inducible factor-1alpha expression in brain development and neonatal hypoxic-ischemic brain injury. Brain Res Rev. Dec. 11, 2009; 62(1):99-108.
47. Quinzanos-Fresnedo J, Coronado-Zarco R, Arch-Tirado E, Verduzco-Mendoza A, Del Valle-Cabrera G, Alfaro-Rodriguez A. [Neurological effects by occlusion of the carotid artery and induced hypoxia in newborn rats]. Cir Cir. March-April 2008; 76(2):119-125.
48. Licht P, Losch A, Dittrich R, Neuwinger J, Siebzehnrubl E, Wildt L. Novel insights into human endometrial paracrinology and embryo-maternal communication by intrauterine microdialysis. Hum Reprod Update. September-October 1998; 4(5):532-538.
49. El Ghazi F, Desfeux A, Brasse-Lagnel C, et al. NO-dependent protective effect of VEGF against excitotoxicity on layer VI of the developing cerebral cortex. Neurobiol Dis. March 2012; 45(3):871-886.
50. Zheng X R, Zhang S S, Yin F, et al. Neuroprotection of VEGF-expression neural stem cells in neonatal cerebral palsy rats. Behav Brain Res. Apr. 21, 2012; 230(1):108-115.
51. Ara J, Fekete S, Frank M, Golden J A, Pleasure D, Valencia I. Hypoxic-preconditioning induces neuroprotection against hypoxia-ischemia in newborn piglet brain. Neurobiol Dis. August 2011; 43(2):473-485.
52. Feng Y, Rhodes P G, Bhatt A J. Neuroprotective effects of vascular endothelial growth factor following hypoxic ischemic brain injury in neonatal rats. Pediatr Res. October 2008; 64(4):370-374.
53. Huang Y F, Zhuang S Q, Chen D P, Liang Y J, Li X Y. [Angiogenesis and its regulatory factors in brain tissue of neonatal rat hypoxic-ischemic encephalopathy]. Zhonghua Er Ke Za Zhi. March 2004; 42(3):210-214.
54. Ran R, Xu H, Lu A, Bernaudin M, Sharp F R. Hypoxia preconditioning in the brain. Dev Neurosci. March-August 2005; 27(2-4):87-92.
55. Sentilhes L, Marret S, Leroux P, Gonzalez B J, Laquerriere A. Vascular-endothelial growth factor and its high affinity receptor VEGFR-2 in the normal versus destructive lesions human forebrain during development: an immuno-histochemical comparative study. Brain Res. Apr. 18, 2011; 1385:77-86.
56. Sivakumar V, Lu J, Ling E A, Kaur C. Vascular endothelial growth factor and nitric oxide production in response to hypoxia in the choroid plexus in neonatal brain. Brain Pathol. January 2008; 18(1):71-85.
57. Tang Y, Pacary E, Freret T, et al. Effect of hypoxic preconditioning on brain genomic response before and following ischemia in the adult mouse: identification of potential neuroprotective candidates for stroke. Neurobiol Dis. January 2006; 21(1):18-28.
58. Zhang S S, Zheng X R, Yang Y J. [Adenovirus-mediated VEGF165 gene transfer has neuroprotective effects in neonatal rats following hypoxic-ischemic brain damage]. Zhongguo Dang Dai Er Ke Za Zhi. December 2008; 10(6):737-742.
59. Shah D K, Doyle L W, Anderson P J, et al. Adverse neurodevelopment in preterm infants with postnatal sepsis or necrotizing enterocolitis is mediated by white matter abnormalities on magnetic resonance imaging at term. J Pediatr. August 2008; 153(2):170-175, 175 e171.
60. Volpe J J. Postnatal sepsis, necrotizing entercolitis, and the critical role of systemic inflammation in white matter injury in premature infants. J Pediatr. August 2008; 153(2):160-163.
61. Procianoy R S, Silveira R C. Association between high cytokine levels with white matter injury in preterm infants with sepsis. Pediatr Crit Care Med. March 2012; 13(2):183-187.
62. Mallard C, Davidson J O, Tan S, et al. Astrocytes and microglia in acute cerebral injury underlying cerebral palsy associated with preterm birth. Pediatr Res. Oct. 31, 2013.
63. Khan N A, Vierboom M P, van Holten-Neelen C, et al. Mitigation of septic shock in mice and rhesus monkeys by human chorionic gonadotrophin-related oligopeptides. Clin Exp Immunol. June 2010; 160(3):466-478.
64. Kim HIM, Rim H K, Shin T, et al. Human chorionic gonadotropin induces nitric oxide synthesis by murine microglia. Int J Immunopharmacol. June 2000; 22(6):453-461.
65. Zhu C, Sun Y, Gao J, Wang X, Plesnila N, Blomgren K. Inhaled Nitric Oxide Protects Males But not Females from Neonatal Mouse Hypoxia-Ischemia Brain Injury. Transl Stroke Res. April 2013; 4(2):201-207.
66. Bonnin P, Leger P L, Villapol S, et al. Dual action of N O synthases on blood flow and infarct volume consecutive to neonatal focal cerebral ischemia. Exp Neurol. July 2012; 236(1):50-57.
67. Colton C A. Induction of nitric oxide in cultured microglia: evidence for a cytoprotective role. Adv Neuroimmunol. 1995; 5(4491-503.

### References for FIG. 4

1. Lorzadeh N, Kazemirad S. The effects of fetal gender on serum human chorionic gonadotropin and testosterone in normotensive and preeclamptic pregnancies. J Pregnancy. 2012; 2012:874290.
2. Thorngren-Jerneck K, Herbst A. Perinatal factors associated with cerebral palsy in children born in Sweden. Obstet Gynecol. December 2006; 108(6):1499-1505.
3. O'Callaghan M E, MacLennan A H, Gibson C S, et al. Epidemiologic associations with cerebral palsy. Obstet Gynecol. September 2011; 118(3):576-582.
4. Muller F, Bussieres L, Pelissier M C, et al. Do racial differences exist in second-trimester maternal hCG levels? A study of 23,369 patients. Prenat Diagn. July 1994; 14(7):633-636.
5. Lang T C, Fuentes-Afflick E, Gilbert W M, Newman T B, Xing G, Wu Y W. Cerebral palsy among Asian ethnic subgroups. Pediatrics. April 2012; 129(4):e992-998.
6. Ball S, Ekelund C, Wright D, et al. Temporal effects of maternal and pregnancy characteristics on serum pregnancy-associated plasma protein-A and free beta-human chorionic gonadotropin at 7-14 weeks' gestation. Ultrasound Obstet Gynecol. Jun. 11, 2012.
7. Kallen A J, Finnstrom O O, Lindam A P, Nilsson E M, Nygren K G, Olausson P M. Cerebral palsy in children born after in vitro fertilization. Is the risk decreasing? Eur J Paediatr Neurol. November 2010; 14(6):526-530.
8. Yigiter A B, Kayak Z N, Bakirci N, Gokaslan H. Effect of smoking on pregnancy-associated plasma protein A, free beta-human chorionic gonadotropin, and nuchal translucency in the first trimester of pregnancy. Advances in therapy. January-February 2006; 23(1):131-138.
9. Gokdeniz R, Ariguloglu E, Bazoglu N, Balat 0. Eleveated Serum B-hCG Levels in Severe Preeclampsia. Turk J Med Sci. 2000; 30:43-45.
10. Hsu C D, Chan D W, Nye B, Johnson T R, Hong S F, Repke J T. Elevated serum human chorionic gonadotropin as evidence of secretory response in severe preeclampsia. Am J Obstet Gynecol. April 1994; 170(4):1135-1138.
11. Xiong X, Saunders L D, Wang F L, Davidge S T, Buekens P. Preeclampsia and cerebral palsy in low-birth-weight and preterm infants: implications for the current “ischemic model” of preeclampsia. Hypertens Pregnancy. 2001; 20(1):1-13.
12. Cole L A. New discoveries on the biology and detection of human chorionic gonadotropin. Reprod Biol EndocrinoL 2009; 7:8.
13. van Schrojenstein Lantman-de Valk H M, van den Akker M, Maaskant M A, et al. Prevalence and incidence of health problems in people with intellectual disability. J Intellect Disabil Res. February 1997; 41 (Pt 1):42-51.
14. Bartels I, Hoppe-Sievert B, Bockel B, Herold S, Caesar J. Adjustment formulae for maternal serum alpha-fetoprotein, human chorionic gonadotropin, and unconjugated oestriol to maternal weight and smoking. Prenat Diagn. February 1993; 13(2):123-130.
15. Prats P, Rodriguez I, Nicolau J, Comas C. Early first-trimester free-beta-hCG and PAPP-A serum distributions in monochorionic and dichorionic twins. Prenat Diagn. January 2012; 32(1):64-69.
16. Scher A I, Petterson B, Blair E, et al. The risk of mortality or cerebral palsy in twins: a collaborative population-based study. Pediatric research. November 2002; 52(5):671-681.

1. A method of treating Cerebral Palsy in a patient or subject in need comprising administering to said patient or subject an effective amount of a compound selected from the group consisting of hCG, a subunit of hCG, a variant of hCG or a mixture thereof.

2. The method according to claim 1 wherein said patient or subject is a neonate, infant or child of an age no greater than about 4 years.

3. The method according to claim 1 wherein said patient or subject is an infant or child of an age no greater than about 2 years.

4. The method according to claim 1 wherein said patient or subject is a neonate.

5. The method according to claim 1 or 4 wherein said patient or subject is a preterm neonate.

6. The method according to any of claims 1-5 wherein said compound is hCG, hyperglycosylated hCG, free β-subunit of hCG or hyperglycosylated free β-subunit hCG.

7. The method according to any of claims 1-6 wherein said compound is hCG.

8. The method according to any of claims 1-6 wherein said compound is hyperglycosylated hCG.

9. The method according to any of claims 1-6 wherein said compound is free β-subunit hCG.

10. The method according to any of claims 1-6 wherein said compound is hyperglycosylated free β-subunit hCG.

11. A method of reducing the likelihood of Cerebral Palsy in a patient or subject at risk for Cerebral Palsy, comprising administering to said patient or subject an effective amount of a compound selected from the group consisting of hCG, a subunit of hCG, a variant of hCG or a mixture thereof.

12. The method according to claim 11 wherein said patient or subject is a neonate, infant or child of an age no greater than about 4 years.

13. The method according to claim 11 wherein said patient or subject is an infant or child of an age no greater than about 2 years.

14. The method according to claim 11 wherein said patient or subject is a neonate.

15. The method according to claim 11 or 14 wherein said patient or subject is a preterm neonate.

16. The method according to claim 11 wherein said patient or subject is a fetus.

17. The method according to claim 16 wherein said compound is administered to the mother who is pregnant with said fetus.

18. The method according to any of claims 11-17 wherein said compound is hCG, hyperglycosylated hCG, free β-subunit of hCG or hyperglycosylated free β-subunit hCG.

19. The method according to any of claims 11-18 wherein said compound is hCG.

20. The method according to any of claims 11-18 wherein said compound is hyperglycosylated hCG.

21. The method according to any of claims 11-18 wherein said compound is free β-subunit hCG.

22. The method according to any of claims 11-18 wherein said compound is hyperglycosylated free β-subunit hCG.

23. A method of upregulating VEGF in a patient or subject in need in an effort to ameliorate the severity of Cerebral Palsy or any of its co-morbidities in said patient or subject comprising administering to said patient or subject an effective amount of a compound selected from the group consisting of hCG, a subunit of hCG, a variant of hCG or a mixture thereof.

24. The method according to claim 23 wherein said patient or subject is a neonate, infant or child of an age no greater than about 4 years.

25. The method according to claim 23 wherein said patient or subject is an infant or child of an age no greater than about 2 years.

26. The method according to claim 23 wherein said patient or subject is a neonate.

27. The method according to claim 23 wherein said patient or subject is a fetus.

28. The method according to claim 27 wherein said compound is administered to the mother who is pregnant with said fetus.

29. The method according to any of claims 23-28 wherein said compound is hCG, hyperglycosylated hCG, free β-subunit of hCG or hyperglycosylated free β-subunit hCG.

30. The method according to any of claims 23-29 wherein said compound is hCG.

31. The method according to any of claims 23-29 wherein said compound is hyperglycosylated hCG.

32. The method according to any of claims 23-29 wherein said compound is free β-subunit hCG.

33. The method according to any of claims 23-29 wherein said compound is hyperglycosylated free β-subunit hCG.

34. A method of inhibiting or ameliorating at least one CP co-morbidity in a patient or subject in need comprising administering to said patient or subject an effective amount of a compound selected from the group consisting of hCG, a subunit of hCG, a variant of hCG or a mixture thereof.

35. The method according to claim 34 wherein said patient or subject is a neonate, infant or child of an age no greater than about 4 years.

36. The method according to claim 34 wherein said patient or subject is an infant or child of an age no greater than about 2 years.

37. The method according to claim 34 wherein said patient or subject is a neonate.

38. The method according to claim 34 wherein said patient or subject is a fetus.

39. The method according to claim 38 wherein said compound is administered to the mother who is pregnant with said fetus.

40. The method according to any of claims 34-39 wherein said compound is hCG, hyperglycosylated hCG, free β-subunit of hCG or hyperglycosylated free β-subunit hCG.

41. The method according to any of claims 34-40 wherein said compound is hCG.

42. The method according to any of claims 34-40 wherein said compound is hyperglycosylated hCG.

43. The method according to any of claims 34-40 wherein said compound is free β-subunit hCG.

44. The method according to any of claims 34-40 wherein said compound is hyperglycosylated free β-subunit hCG.

45. The method according to any of claims 34-40 wherein said co-morbidity of Cerebral Palsy is chronic cognitive impairment, expressive and/or receptive language impairment, partial or complete hearing or vision loss, seizures (Epilepsy), chronic pain, behavior difficulties, neurobehavioral disorders such as attention deficits and hyperactivity, Autism Spectrum Disorder, eating and digestive co-morbidities, increased drooling, slow growth, irregular breathing and/or urinary incontinence.

46. A method of treating white matter injury or reducing the likelihood that white matter injury will result in Cerebral Palsy in a patient or subject in need comprising administering to said patient or subject an effective amount of a compound selected from the group consisting of hCG, a subunit of hCG, a variant of hCG or a mixture thereof.

47. The method according to claim 46 wherein said patient or subject is a neonate, infant or child of an age no greater than about 4 years.

48. The method according to claim 46 wherein said patient or subject is an infant or child of an age no greater than about 2 years.

49. The method according to claim 46 wherein said patient or subject is a neonate.

50. The method according to claim 46 wherein said patient or subject is a fetus.

51. The method according to claim 50 wherein said compound is administered to the mother who is pregnant with said fetus.

52. The method according to any of claims 46-51 wherein said compound is hCG, hyperglycosylated hCG, free β-subunit of hCG or hyperglycosylated free β-subunit hCG.

53. The method according to any of claims 46-52 wherein said compound is hCG.

54. The method according to any of claims 46-52 wherein said compound is hyperglycosylated hCG.

55. The method according to any of claims 46-52 wherein said compound is free β-subunit hCG.

56. The method according to any of claims 46-52 wherein said compound is hyperglycosylated free β-subunit hCG.

57. The method according to any of claims 46-56 wherein said white matter injury is leukomalacia.

58. The method according to any of claims 46-56 wherein said white matter injury is present is a neonate, infant or child less than about 4 years of age.

59. Use of a compound selected from the group consisting of hCG, a subunit of hCG, a variant of hCG or a mixture thereof in the manufacture of a medicament for the treatment of Cerebral Palsy in a subject or patient.

60. Use according to claim 59 wherein said compound is hCG, hyperglycosylated hCG, free β-subunit of hCG or hyperglycosylated free β-subunit hCG.

61. Use of a compound selected from the group consisting of hCG, a subunit of hCG, a variant of hCG or a mixture thereof in the manufacture of a medicament for reducing the likelihood of Cerebral Palsy in a subject or patient at risk for Cerebral Palsy.

62. Use according to claim 61 wherein said compound is hCG, hyperglycosylated hCG, free β-subunit of hCG or hyperglycosylated free β-subunit hCG.

63. Use of a compound selected from the group consisting of hCG, a subunit of hCG, a variant of hCG or a mixture thereof in the manufacture of a medicament for inhibiting or ameliorating at least one co-morbidity of Cerebral Palsy in a patient or subject in need.

64. Use according to claim 63 wherein said compound is hCG, hyperglycosylated hCG, free β-subunit of hCG or hyperglycosylated free β-subunit hCG.

65. Use according to claim 63 or 64 wherein said co-morbidity of Cerbral Palsy is chronic cognitive impairment, expressive and/or receptive language impairment, partial or complete hearing or vision loss, seizures (Epilepsy), chronic pain, behavior difficulties, neurobehavioral disorders such as attention deficits and hyperactivity, Autism Spectrum Disorder, eating and digestive co-morbidities, increased drooling, slow growth, irregular breathing and/or urinary incontinence.

66. Use of a compound selected from the group consisting of hCG, a subunit of hCG, a variant of hCG or a mixture thereof in the manufacture of a medicament for treating white matter injury or reducing the likelihood that white matter injury will result in Cerebral Palsy in a patient or subject in need.

67. Use according to claim 66 wherein said compound is hCG, hyperglycosylated hCG, free β-subunit of hCG or hyperglycosylated free β-subunit hCG.

68. Use according to claim 66 or 67 wherein said white matter injury is leukomalacia.

69. Use according to claim 66 or 67 wherein said white matter injury is present in a neonate, infant or child less than about 4 years of age.
